(19) United States
(12) Patent Application Publication
(10) Pub. No.: US 2023/0070258 A1
(43) Pub. Date: Mar. 9, 2023

(54) COMPOSITIONS AND METHODS FOR THE TREATMENT OF RHEUMATOID ARTHRITIS

(71) Applicant: Navidea Biopharmaceuticals, Inc., Dublin, OH (US)

(72) Inventors: David A. Ralph, Columbus, OH (US); Michael Rosol, Dublin, OH (US)

(21) Appl. No.: 17/891,450

(22) Filed: Aug. 19, 2022

Related U.S. Application Data
(60) Provisional application No. 63/235,080, filed on Aug. 19, 2021.

(51) Int. Cl. C07K 16/24; A61K 51/04; A61P 19/02; G16H 50/50; G16H 30/40; G06T 7/00

(52) U.S. Cl. CPC C07K 16/241; A61K 51/0497; A61P 19/02; G16H 50/50; G16H 30/40; G06T 7/0014; A61K 2039/505

(57) ABSTRACT

Disclosed herein are methods of treating a subject with rheumatoid arthritis (RA) and methods of predicting the subject's likelihood of responding to a new RA therapy. The methods include administering to the subject a composition comprising a macrophage targeting construct and an imaging moiety conjugated thereto to acquiring planar images of a plurality of joints of the subject, and determining at least one TUV_{Global} value for the subject from the planar images. Covariates comprising the quantification of serological RA markers and/or clinical assessments may be further obtained to apply statistical modeling to the combination of the at least one TUV_{Global} and the one or more covariates. The statistical modeling is used to determine a likelihood of response to an RA therapy, and a treatment is administered to the subject based on the likelihood of response to the RA therapy.

### CROSS-REFERENCE TO RELATED APPLICATIONS

This application claims priority and is related to U.S. Provisional Application Ser. No. 63/235,080 filed on Aug. 19, 2021 and entitled Compositions and Methods for the Treatment of Rheumatoid Arthritis. The entire contents of this patent application are expressly incorporated herein by reference including, without limitation, the specification, claims, and abstract, as well as any figures, tables, or drawings thereof.

### BACKGROUND

Rheumatoid arthritis (RA) affects approximately 1% of the world's population and is characterized by inflammation and cellular proliferation in the synovial lining of joints, often resulting in cartilage and bone destruction, joint deformity, and loss of mobility. RA therapies benefit some, but not all patients with RA, and it can frequently take many months to determine which patients will benefit from a given course of therapy. This results in patients having to take RA therapies for months before determining whether or not an RA therapy is effective. For patients who do not respond well to the RA therapy administered, this results in months of delay in achieving symptomatic relief and adequate treatment, and increasing costs to the patient.

The current standard of care for determining if a patient is responding to a new RA therapy is to first determine the severity of their RA disease prior to the administration of RA therapy (i.e., at TO), and then again after 6 months of RA therapy using the American College of Rheumatology/European League Against Rheumatism 2010 criteria (ACR criteria score). The ACR criteria score is a measure of RA disease activity. A patient's response to RA therapy is measured by how much their ACR criteria score declines between T0 and their 6-month clinical assessments. For example, an ACR20, ACR50, and ACR70 response corresponds to a 20%, 50%, and 70% decline in their ACR criteria score respectively. As such, with the current standard of care, a patient must wait 6 months before identifying whether or not the RA therapy is effective.

Accordingly, there is a need in the art to determine more quickly which patients are likely to respond to a therapy so that non-responders can be switched to alternative therapies.

### BRIEF SUMMARY OF THE INVENTION

Disclosed herein are methods of treating a subject with RA and predicting a subject's likelihood of response to a new RA therapy. In Example 1, a method of treating a subject with rheumatoid arthritis (RA) comprises (a) administering to the subject a composition comprising a macrophage targeting construct and an imaging moiety conjugated thereto; (b) acquiring planar images of a plurality of joints of the subject; (c) determining at least one TUV_{Global} value for the subject from the planar images; (d) obtaining one or more covariates comprising: (i) a serological covariate obtained from a serum sample from the subject and quantifying the level of one or more RA markers in the serum; and/or (ii) a clinical covariate obtained from results of one or more clinical assessment tests; (e) applying statistical modeling to the at least one TUV_{Global} and the one or more covariates to determine a likelihood of response to an RA therapy; and (f) administering a treatment to the subject based on the likelihood of response to the RA therapy.

Example 2 relates to the method according to Example 1, wherein the RA therapy comprises an anti-TNF (aTNF) therapy, anti-IL6 therapy, anti-IL1 therapy, anti-CD20 therapy, anti-GM-CSF therapy, CTLA4-based therapy, JAK inhibitors, or a combination thereof.

Example 3 relates to the method according to Example 2, wherein the treatment comprises the RA therapy or a therapy that is not the RA therapy.

Example 4 relates to the method according to Example 1, wherein the RA therapy comprises an aTNF therapy, and wherein the treatment comprises the aTNF therapy or a non-aTNF therapy.

Example 5 relates to the method according to Example 1, wherein the serological covariate comprises C-Reactive Protein (CRP), Rheumatoid Factor (RF), Erythrocyte Sedimentation Rate (ESR), or anti-citrullinated peptide antibodies (ACPA).

Example 6 relates to the method according to Example 1, wherein the clinical covariate comprises the Health Assessment Questionnaire—Disease Index (HAQ-DI), Clinical Disease Activity Index (CDAI), Disease Activity Score of 28 Joints (DAS), or Visual Analog Scale (VAS).

Example 7 relates to the method according to Example 5, wherein the CRP, RF, ESR, and ACPA are obtained.

Example 8 relates to the method according to Example 6, wherein the HAQ-DI, CDAI, DAS, and VAS are obtained.

Example 9 relates to the method according to Example 1, wherein at least one serological covariate and at least one clinical covariate are obtained.

Example 10 relates to the method according to Example 1, wherein the at least one TUV_{Global} value is determined prior to the administration of the RA therapy.

Example 11 relates to the method according to Example 1, wherein the at least one TUV_{Global} value is determined at a time period between one week and 24 weeks after the administration of the RA therapy.

Example 12 relates to the method according to Example 1, wherein the likelihood of treatment response is the likelihood that the RA therapy results in an at least 20% reduction in an ACR criteria score (American College of Rheumatology/European League Against Rheumatism 2010 criteria) of the subject at about 24 weeks after the administration of the RA therapy.

Example 13 relates to the method according to Example 2, wherein the RA therapy administered is the aTNF therapy and results in an at least 50% reduction in an ACR criteria score (American College of Rheumatology/European League Against Rheumatism 2010 criteria) of the subject at about 24 weeks after the administration of the RA therapy.

Example 14 relates to the method according to Example 2, wherein the RA therapy administered is the aTNF therapy and results in an at least 70% reduction in an ACR criteria score (American College of Rheumatology/European League Against Rheumatism 2010 criteria) of the subject at about 24 weeks after the administration of the RA therapy.

Example 15 relates to the method according to Example 1, wherein the statistical modeling comprises a logistic regression model.

Example 16 relates to the method of Example 1, wherein the step of determining the TUV_{Global} value further comprises (a) selecting a plurality of joints in the subject where inflammation is suspected; (b) acquiring one or more planar images of each of the plurality of joints; (c) for each joint image, defining an ROI comprising the joint; (d) for each joint, defining a joint specific RR; (e) for each joint, determining a TUV_{Joint} value of the joint by assessing the ratio of average pixel intensity of the ROI to the average pixel intensity of the RR; (f) for each joint, comparing the TUV_{Joint} value of the joint to a normal TUV_{Joint} value for a corresponding joint, wherein the normal TUV_{Joint} value is derived from averaging the TUV_{Joint} values for the corresponding joint from a plurality of healthy subjects, and wherein macrophage involvement is indicated by a joint specific TUV_{Joint} value that exceeds the normal TUV_{Joint} value by a predetermined threshold; (g) for each joint having a joint specific TUV_{Joint} value that exceeds the normal TUV_{Joint} value by a predetermined threshold, calculating a macrophage-involved contribution (MI) of the joint by dividing the difference of the TUV_{Joint} and normal TUV_{Joint} by the normal TUV_{Joint}; and (h) determining the TUV_{Global} value for the subject by determining the sum of the MI for all of the joints of the subject that exceeds the predetermined threshold.

Example 17 relates to the method according to Example 1, wherein the macrophage targeting construct is a mannosylated dextran construct comprising Tc99m-tilmanocept, and wherein the quantity of Tc99m-tilmanocept administered is between about 50 μg and about 400 μg.

Example 18 relates to the method according to Example 1, wherein the subject is initiating a new RA therapy.

Example 19 relates to the method according to Example 18, wherein the method is performed prior to the subject initiating a new RA therapy.

In Example 20, a method of predicting a subject's likelihood of response to a new RA therapy comprises (a) administering to the subject a composition comprising a macrophage targeting construct and an imaging moiety conjugated thereto; (b) acquiring planar images of a plurality of joints of the subject; (c) determining at least one TUV_{Global} value for the subject from the planar images; (d) obtaining one or more covariates comprising: (i) a serological covariate obtained from a serum sample from the subject and quantifying the level of one or more RA markers in the serum; and/or (ii) a clinical covariate obtained from results of one or more clinical assessment tests; and (e) applying statistical modeling to the at least one TUV_{Global} and the one or more covariates to determine the likelihood of treatment response to a new anti-TNF (aTNF) therapy.

Example 21 relates to the method according to Example 20, wherein the serological covariate comprises C-Reactive Protein (CRP), Rheumatoid Factor (RF), Erythrocyte Sedimentation Rate (ESR), or anti-citrullinated peptide antibodies (ACPA), wherein the clinical covariate comprises the Health Assessment Questionnaire—Disease Index (HAQ-DI), Clinical Disease Activity Index (CDAI), Disease Activity Score of 28 Joints (DAS), or Visual Analog Scale (VAS), and wherein at least one serological covariate and at least one clinical covariate are obtained.

Example 22 relates to the method according to Example 20, wherein the statistical modeling comprises a logistic regression model.

While multiple embodiments are disclosed, still other embodiments of the present disclosure will become apparent to those skilled in the art from the following detailed description, which shows and describes illustrative embodiments of the disclosure. Accordingly, the detailed description is to be regarded as illustrative in nature and not restrictive.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, PA, and 4% This applies regardless of the breadth of the range.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the disclosure.

As used herein, the term “pharmaceutically acceptable carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “subject” or “patient” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with rheumatoid arthritis” means having been subjected to a physical examination by a person of skill, for example, a physician, and found to have a condition that can be diagnosed or treated by a compound or composition that can reduce inflammation of the joints and/or the pain associated therewith.

The term “subject with RA” refers to a subject that presents one or more symptoms indicative of RA (e.g., pain, stiffness or swelling of joints), or that is screened for RA (e.g., during a physical examination). Alternatively, or additionally, a subject suspected of having RA may have one or more risk factors (e.g., age, sex, family history, smoking, etc). The term encompasses subjects that have not been tested for RA as well as subjects that have received an initial diagnosis.

As used herein, the terms “administering”, and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Tilmanocept” refers to a non-radiolabeled precursor of the LYMPHOSEEK® diagnostic agent. Tilmanocept is a mannosylaminodextran. It has a dextran backbone to which a plurality of amino-terminated leashes (—O(CH₂)₃S(CH₂)₂NH₂) are attached to the core glucose elements. In addition, mannose moieties are conjugated to amino groups of a number of the leashes, and the chelator diethylenetriamine pentaacetic acid (DTPA) may be conjugated to the amino group of other leashes not containing the mannose. Tilmanocept generally, has a dextran backbone, in which a plurality of the glucose residues comprise an amino-terminated leash:

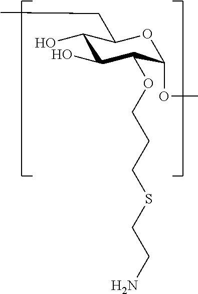

the mannose moieties are conjugated to the amino groups of the leash via an amidine linker:

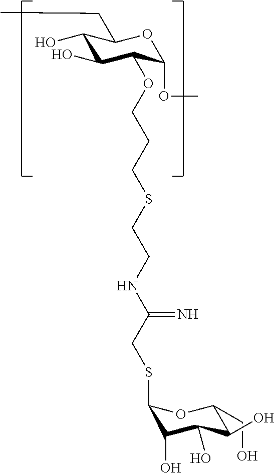

the chelator diethylenetriamine pentaacetic acid (DTPA) is conjugated to the amino groups of the leash via an amide linker:

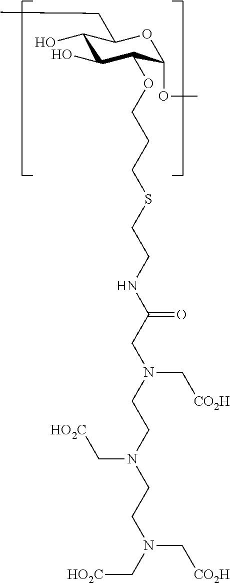

Tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, and tilmanocept Tc99m has the following molecular formula: [C₆H₁₀O₅]ₙ·(C₁₉H₂₈N₄O₉S^{99m}Tc)_{b}·(C₁₃H₂₄N₂O₅S₂)c·(C₅H₁₁NS)_{α} and contains 3-8 conjugated DTPA molecules (b); 12-20 conjugated mannose molecules (c); and 0-17 amine side chains (a) remaining free. Tilmanocept has the following general structure:

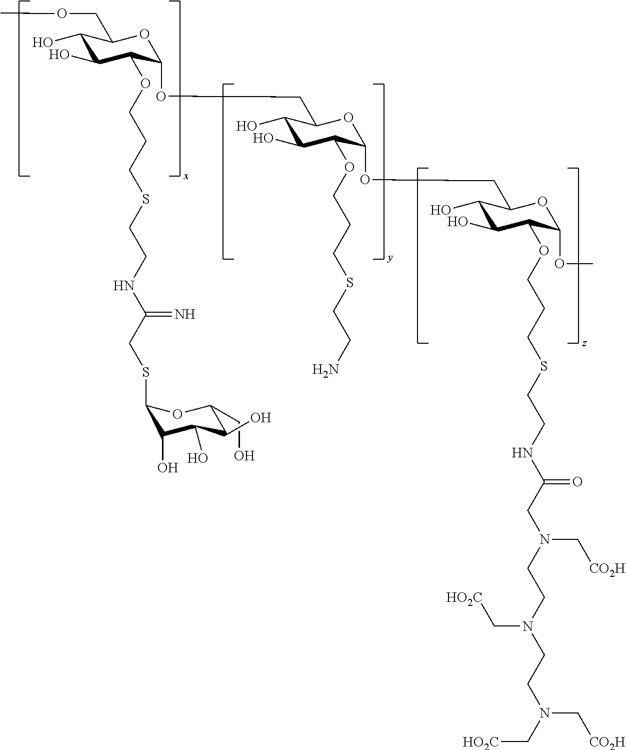

Certain of the glucose moieties may have no attached amino-terminated leash.

The terms “anti-TNF therapy” or “aTNF therapy” as used herein are intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or nonnaturally-occurring molecules, and/or recombinant and/or engineered forms thereof, that, directly or indirectly, inhibits TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signaling or any other means resulting in decreased TNFα activity in a subject. The term “TNFα inhibitor” also includes agents which interfere with TNFα activity. Examples of TNFα inhibitors include etanercept (ENBREL®, Amgen), infliximab (REMICADE®, Johnson and Johnson), human anti-TNF monoclonal antibody adalimumab (D2E7/HUMIRA®, Abbott Laboratories), golimumab (SIMPONI®/SIMPONI ARIA®, Janssen Biotech, Inc.), certolizumab (CIMZIA®, UCB, Inc.), CDP 571 (Celltech), and CDP 870 (Celltech), as well as other compounds which inhibit TNFα activity, such that when administered to a subject suffering from or at risk of suffering from a disorder in which TNFα activity is detrimental (e.g., RA), the disorder is treated.

A “non-aTNF therapy” can be any treatment known in the art to be effective for the treatment of RA that does not involve antagonism of TNFα for its mechanism of action. For example, the conventionally well-known therapeutic drugs include biological preparations, non-steroidal anti-inflammatory drugs (anti-inflammatory analgesics), steroidal drugs and immunosuppressants. The non-steroidal anti-inflammatory drugs include prostaglandin synthesis inhibitors. Additional non-aTNF therapies may include anti-IL6 therapy, anti-IL1 therapy, anti-CD20 therapy, anti-GM-CSF therapy, CTLA4-based therapy, JAK inhibitors, or a combination thereof.

Disclosed herein are methods of treating a subject with RA by providing an early indication of the likelihood of response to a newly initiated RA therapy. In some aspects, the early indication is determined by combining information from analyses of imaging results assessing macrophage involvement in RA pathobiology (i.e. TUV_{Global} values) with clinical assessments and serological results obtained prior to initiation of RA therapy. In some embodiments, the assessment is done utilizing a logistic regression statistical model.

Disclosed herein are methods of treating a subject with RA comprising administering to the subject a composition comprising a macrophage targeting construct and an imaging moiety conjugated thereto, acquiring planar images of a plurality of joints of the subject; determining at least one TUV_{Global} value for the subject from the planar images; obtaining one or more covariates comprising, (i) a serological covariate obtained from a biological sample (e.g. a serum sample) from the subject and quantifying the level of one or more RA markers in the sample; and/or (ii) a clinical covariate obtained from results of one or more clinical assessment tests; applying statistical modeling to the at least one TUV_{Global} and the one or more covariates to determine a likelihood of treatment response to a RA therapy; and administering a treatment to the subject based on the likelihood of treatment response to the RA therapy. Further disclosed herein is a method to quantify the amount of macrophage involved disease activity in a particular anatomical region of interest and to quantitatively determine how macrophage involvement changes over time and in response to therapies.

Further disclosed herein are methods for evaluating a subject with RA initiating a new therapy or treatment for RA and methods of predicting the likelihood that the subject will respond to the new therapy or treatment. In implementations, the new therapy or treatment may comprise an RA therapy comprising aTNF therapy, anti-IL6 therapy, anti-IL1 therapy, anti-CD20 therapy, anti-GM-CSF therapy, CTLA4-based therapies (such as, for example, abatacept), JAK inhibitors, or a combination thereof. In some implementations, the disclosed methods are useful in determining whether a subject with RA will be likely to respond to a new RA therapy. In further embodiments, the disclosed methods are useful to determine whether a subject with RA is likely to fail to respond to a new RA therapy. In yet further embodiments, the disclosed methods are useful in allowing for early termination of a new RA therapy or treatment in a subject with RA by determining that further treatment is likely to fail, and alternatively administering a treatment that is not the RA therapy that is determined likely to fail to the subject. As used herein “early termination” means the treatment is terminated substantially earlier than it would be under standard clinical practice (e.g. about 5 weeks).

### Macrophage Targeting Construct

In certain aspects, the compounds disclosed herein employ a carrier construct comprising a macrophage targeting construct. The macrophage targeting construct may comprise any construct having the capability of binding to macrophages. Examples of suitable macrophage targeting constructs include, but are not limited to, mannosylated dextran constructs, somatostatin receptor ligands (including, but not limited to, DOTA-TATE), translocator protein ligands including TSPO, SIGLEC (sialic acid-binding immunoglobulin-type lectins) receptor ligands, antibodies, nanobodies, or fragments thereof that have specificity for CD206, CD163, CD68 or other macrophage specific surface markers, or imaging agents that measure cellular respiration rates, such as, but not limited to, ¹⁸F-labeled fluorodeoxyglucose ([18F]FDG). In some implementations, the macrophage targeting construct may comprise a polymeric (e.g. carbohydrate) backbone having conjugated thereto mannose-binding C-type lectin receptor targeting moieties (e.g. mannose) to deliver one or more active therapeutic agents. Examples of such constructs include mannosylamino dextrans (MAD) or mannosylated dextran constructs, which comprise a dextran backbone having mannose molecules conjugated to glucose residues of the backbone and having an active pharmaceutical ingredient conjugated to glucose residues of the backbone. Tilmanocept is a specific example of an MAD. A tilmanocept derivative that is tilmanocept without DTPA conjugated thereto is a further example of an MAD.

In certain implementations, the disclosure provides a compound comprising a dextran-based moiety or backbone having one or more mannose-binding C-type lectin receptor targeting moieties and one or more therapeutic agents attached thereto. The dextran-based moiety generally comprises a dextran backbone similar to that described in U.S. Pat. No. 6,409,990 (the '990 patent), which is incorporated herein by reference. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, mannose-binding C-type lectin receptor targeting moieties are attached to between about 10% and about 50% of the glucose residues of the dextran backbone, or between about 20% and about 45% of the glucose residues, or between about 25% and about 40% of the glucose residues.

According to further aspects, the mannose-binding C-type lectin receptor targeting moiety is selected from, but not limited to, mannose, fucose, and n-acetylglucosamine. In some embodiments, the targeting moieties are attached to between about 10% and about 50% of the glucose residues of the dextran backbone, or between about 20% and about 45% of the glucose residues, or between about 25% and about 40% of the glucose residues. MWs referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.

According to certain embodiments, the one or more mannose-binding C-type lectin receptor targeting moieties and one or more detectable agents (e.g. a radiolabeled imaging moiety) are attached to the dextran-based moiety by way of a leash. The leash may be attached at from about 50% to about 100% of the backbone moieties or about 70% to about 90%. The leashes may be the same or different. In some embodiments, the leash is an amino-terminated leash or amine-terminated leash. In some embodiments, the leashes may comprise —O(CH₂)₃S(CH₂)₂NH—. In some embodiments, the leash may be a chain of from about 1 to about 20 member atoms selected from carbon, oxygen, sulfur, nitrogen and phosphorus. The leash may be a straight chain or branched. The leash may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; ═N— alkyl; —SH; —S-alkyl; —NH—C(O)—; —NH—C(═N)— and the like. As would be apparent to one skilled in the art, other suitable leashes are possible.

The disclosed compounds can include an imaging moiety or detectable label. As used herein, the term “imaging moiety” means an atom, isotope, or chemical structure which is: (1) capable of attachment to the carrier molecule; (2) non-toxic to humans or other mammalian subjects; and (3) provides a directly or indirectly detectable signal, particularly a signal which not only can be measured but whose intensity is related (e.g., proportional) to the amount of the imaging moiety. The signal may be detected by any suitable means, including spectroscopic, electrical, optical, magnetic, auditory, radio signal, or palpation detection means.

Imaging moieties include, but are not limited to, radioactive isotopes (radioisotopes), fluorescent molecules (a.k.a. fluorochromes and fluorophores), chemiluminescent reagents (e.g., luminol), bioluminescent reagents (e.g., luciferin and green fluorescent protein (GFP)), and metals (e.g., gold nanoparticles). Suitable imaging moieties can be selected based on the choice of imaging method. For example, the detection label can be a near infrared fluorescent dye for optical imaging, a Gadolinium chelate for Mill imaging, a radionuclide for PET or SPECT imaging, or a gold nanoparticle for CT imaging.

Imaging moieties can be selected from, for example, a radionuclide, a radiological contrast agent, a paramagnetic ion, a metal, a fluorescent label, a chemiluminescent label, an ultrasound contrast agent, a photoactive agent, or a combination thereof. Non-limiting examples of imaging moieties include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸G, ³²P, ¹¹C, ¹³N, ¹⁵O, ¹⁸⁹Re, ¹⁸⁸Re, ⁵¹Mn, ^{52m}Mn, ⁵⁵Co, ⁷²As, ⁷⁶Br, ^{82m}Rb, ⁸³Sr, ^{117m}Sn or other gamma-, beta-, or positron-emitters. Gamma radiation from radioisotopes can be detected using a gamma particle detection device. In some embodiments, the gamma particle detection device is a Gamma Finder® device (SenoRx, Irvine Calif.). In some embodiments, the gamma particle detection device is a Neoprobe® GDS gamma detection system (Dublin, Ohio).

Paramagnetic ions of use may include chromium (III), manganese (II), iron (H), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas-filled liposomes.

Other suitable labels include, for example, fluorescent labels (such as GFP and its analogs, fluorescein, isothiocyanate, rhodamine, phycoerythrin, phycocyanin, allophycocyanin, o-phthaldehyde, and fluorescamine and fluorescent metals such as Eu or others metals from the lanthanide series), near IR dyes, quantum dots, phosphorescent labels, chemiluminescent labels or bioluminescent labels (such as luminal, isoluminol, theromatic acridinium ester, imidazole, acridinium salts, oxalate ester, or dioxetane).

In certain aspects, the mannosylated dextran construct is Tc99m-tilmanocept. In aspects, Tc99m-tilmanocept binds to the mannose receptor (CD206) that is expressed by various myeloid lineage immune cells. Non-limiting examples of myeloid lineage immune cell types that express CD206 include a significant portion of dendritic cells, myeloid derived suppressor cells, and macrophages. Of particular significance is the high level of expression of CD206 on a significant portion of activated macrophages at sites of inflammation. In some aspects, sites of inflammation with high densities of CD206 expressing (CD206+) macrophages include, but are not limited to, a significant portion of the skeletal joints of the hands and wrists that are involved in RA mediated inflammation.

In exemplary implementations of these embodiments, the intended route of administration for Tc-99m tilmanocept is intravenous (IV). In some embodiments the site of IV placement is the left or right antecubital vein. In some embodiments, the IV placement site is between the elbow and wrist. In some embodiments, the quantity of tilmanocept administered IV is between about 50 μg and about 400 μg. In some embodiments, the Tc-99m radiolabeling ranges from about 1 mCi to about 10 mCi. In some embodiments, the Tc-99m tilmanocept is administered IV in one dose. In some embodiments, the Tc-99m tilmanocept is administered IV in more than one dose. In some embodiments, following administration of the Tc-99m tilmanocept, sterile saline is administered. In further aspects, the time period between Tc-99m tilmanocept administration and obtaining the image of the subject is from about 15 minutes to about 6 hours.

In some examples, tilmanocept is labeled with^{99m}technetium [Tc99m]. However, in other examples, tilmanocept and/or modest chemical derivatives of tilmanocept may be labeled with various alternative radioisotopes for planar gamma imaging, single-photon emission computerized tomography (SPECT), or positron emission tomography (PET).

### Acquiring One or More Planar Images

According to certain embodiments, one or more planar images are acquired after a defined time interval following administration of the macrophage targeting construct. In some embodiments, the time between administration and acquisition of images is between about 15 minutes to about 6 hours. In some embodiments, the time between administration and acquisition of images is between about 15 minutes to about 3 hours. In some embodiments, the time between administration and acquisition of images is between about 1 hour to about 3 hours or more. In some embodiments, the time between administration and acquisition of images is between about 4 hours to about 6 hours or more.

In some embodiments, the camera used to acquire planar images for analysis is a dual-headed SPECT or SPECT/CT camera equipped with a low-energy, high-resolution collimator with a 15% window (20% can be used if 15% setting not available), and in certain implementations where Tc99m-tilmanocept is administered, centered over a 140 keV peak. In some embodiments, a target of 5-7 million counts is obtained using state-of-the-art 2-headed cameras (nominal 20″×15″ FOV). According to further implementations, a single headed camera is used for image acquisition. According to certain alternative embodiments, image acquisition period is based on time rather than counts. In exemplary implementations, image acquisition occurs during a window of, for example, about 5 to about 20 minutes. Shorter or longer time periods are possible. In certain embodiments, whole body scans are performed. In further embodiments, only the hands, only the feet, or only the hands and feet are scanned. In the foregoing embodiments, where only the hands and/or feet are scanned, image acquisition time periods are generally a shorter duration than when the whole body is scanned.

### Defining ROI

According to certain embodiments, following image acquisition, one or more regions of interest (ROI) are defined. In certain aspects, the ROI is a subset of the pixels of the full image that contains the anatomical region to be assessed (e.g., a joint). In certain embodiments, an ROI is defined by a health care provider. In alternative embodiments, the ROI is defined by, or with the assistance of a computer implemented algorithm. In exemplary aspects of these embodiments, the algorithm my employ machine learning to improve accuracy of ROI selection.

In some embodiments, intermeans thresholding is used for selecting the ROI. In some embodiments, the ROI is selected manually by drawing an area. In some embodiments, for example in RA, the ROI is manually drawn around a joint. In some embodiments, manual ROIs are drawn tightly around the joint to minimize potential signal dilution from extraneous soft tissue. From these ROIs, the average and/or maximum pixel intensity is obtained, which represents the quantification of disease-specific activated macrophage activity within the ROI.

Several commercial and open-source packages are available for the quantitation of medical images. For example, Image) is an open-architecture, Java-based program developed by the National Institutes of Health (NIH) compatible with Macintosh, Linux, and Windows operating systems. It is equipped with processing features including the calculation of area and pixel value statistics from defined regions, image windowing (i.e., adjust brightness/contrast) for greater visualization without modifying true quantitative data, and the ability to cut, copy, or paste images or selections. ImageJ can open and save a variety of image file extensions including DICOM (Digital Imaging and Communications in Medicine) images.

### Defining RR

In certain aspects, the disclosed method comprises defining a refence region (RR). In exemplary embodiments, reference region is a joint-specific reference region. That is, the reference region selected is matched specifically to the ROI in terms of anatomical proximity and/or size. According to certain implementations, the joint-specific RR is adjacent to the ROI. In further implementations, the RR is approximately adjacent to the ROI. In exemplary implementations of these embodiments, the RR is about 3 ROI diameters or less from the closest edge of the ROI. In further implementations, the RR is about 2 ROI diameters or less from the closest edge of the ROI.

According to certain embodiments, the joint-specific reference region is the same size, or substantially the same size, as the ROI.

According to certain alternative embodiments, and specifically, certain embodiments where the ROI is one or more of the joints of the hands or feet, the RR is defined as an area containing multiple joints of the hands or feet, less the pixel intensity value of the ROIs within the RR. In some implementations of these embodiments, the RR comprises the entire hand and/or wrist, minus the pixel intensity value of the ROIs within the RR. According to certain implementations of these embodiments, the RR is defined as the entire hand and/or wrist, minus the pixel intensity of the MCPs and PIPs. In further embodiments, the RR is defined as an area containing a subset of MCPs and/or PIPs, minus the pixel intensities of the MCPs and PIPs contained with the RR area. In certain implementations, these larger MCP specific reference regions may have less observational variation relative to the individual MCPs than smaller joint specific reference regions. Similar joint type specific reference regions could be drawn for the PIP and wrist joint classes.

### Determining TUV

In certain aspects, the pixel intensities of the ROI and RR are used to derive normalized region-specific mannose receptor binding of the mannosylated dextran construct, referred to herein as TUV. As referred to herein, the terms “TUV” and “MARTAD” may be used interchangeably throughout the disclosure. The TUV value is a quantitative index of the amount of imaging agent localization that can be attributable to disease activity in planar images. Defined broadly, the TUV value is determined by assessing the ratio of average pixel intensity of the ROI to the average pixel intensity of the RR. In certain alternative embodiments, the TUV value is determined by assessing the ratio of maximum pixel intensity of the ROI to the maximum pixel intensity of the RR.

Pixel intensity determinations can be made through many commercial and open-source packages available for the quantitation of medical images known in the art. For example, the RadiAnt DICOM viewer software (v. 5.0.2). Alternatively, the Image) program can be used to quantify ROIs and RRs and summarizes area and intensity values as pixel statistics. These pixel statistics include pixel area, mean intensity, minimum intensity, maximum intensity, and median intensity of the ROI and/or RR.

Determining TUV_{Joint} and TUV_{Global}

Following TUV value determination for a joint or plurality of joints, the TUV_{Joint} value is determined by comparing the TUV of the first joint to a normal TUV value for a corresponding joint (e.g., RtPIP2 RA vs RtPIP2 healthy). In certain implementations, the normal TUV value is determined by aggregating the TUV values for each joint from a pool of healthy subjects (e.g., not suffering from RA). In exemplary implementations, the method for defining the joint specific RR in the pool of healthy subjects will be the same as that used for the patient population. Macrophage involvement in a subject is indicated by a TUV_{Joint} value that exceeds the normal TUV value by a predetermined threshold. In certain implementations, the predetermined threshold is exceeded when the subject TUV_{Joint} value is greater than or equal to two standard deviations of the average TUV value of the corresponding joint from the plurality of healthy subjects. In certain alternative implementations, the predetermined threshold subject TUV_{Joint} value is equal to or greater than the 95% confidence interval of the average MARTAD value of the corresponding joint from the plurality of healthy subjects.

According to certain embodiments, the method further includes determining a TUV_{Global} value of the subject. In certain implementations, the TUV_{Global} value is determined by selecting a plurality of joints in the subject where inflammation is suspected; acquiring one or more planar images of each of the plurality of joints; for each joint image, defining an ROI comprising the joint; for each joint, defining a joint specific RR; for each joint, determining a TUV_{Joint} value of the joint by assessing the ratio of average pixel intensity of the ROI to the average pixel intensity of the RR; for each joint, comparing the TUV_{Joint} value of the joint to a normal TUV_{Joint} value for a corresponding joint, wherein the normal TUV_{Joint} value is derived from averaging the TUV_{Joint} values for the corresponding joint from a plurality of healthy subjects, and wherein macrophage involvement is indicated by a joint specific TUV_{Joint} value that exceeds the normal TUV_{Joint} value by a predetermined threshold; for each joint having a joint specific TUV_{Joint} value that exceeds the normal TUV_{Joint} value by a predetermined threshold, calculating a macrophage-involved contribution (MI) of the joint by dividing the difference of the TUV_{Joint} and normal TUV_{Joint} by the normal TUV_{Joint}; and determining the TUV_{Global} value for the subject by determining the sum of the MI for all of the joints of the subject that exceeds the predetermined threshold.

According to further aspects, planar images comprising at least an anterior image and a posterior image of a joint and its joint specific reference region are evaluated. In exemplary aspects of these embodiments, the subject's TUV value is determined by averaging the TUV values determined from the anterior and posterior images. In further exemplary aspects, TUV values are calculated for all evaluated joints for both the anterior and posterior views with the higher TUV value accepted for further analyses. In further exemplary aspects, for each joint with a TUV value that is within 20% of the predetermined threshold using a single planar image, the TUV value is recalculated using an anterior and posterior planar image.

### Serological and Clinical Covariates

The instantly disclosed methods involve the step of obtaining one or more covariates for use in a statistical model. In embodiments, the one or more covariates are obtained in addition to the TUV values. In aspects, the one or more covariates comprise a serological covariate and/or a clinical covariate.

In some embodiments, the one or more covariates comprise a serological covariate/serological marker. In aspects, the serological covariate may be obtained by collecting a biological sample from the subject for the purpose of analyzing one of more RA markers in the biological sample. In some aspects, the collection step may be applied to any type of biological sample allowing one or more biomarkers to be assayed. Examples of suitable biological samples include, but are not limited to, whole blood, serum, plasma, saliva, and synovial fluid. Biological samples used in the disclosed methods may be fresh or frozen samples collected from a subject, or archival samples with known diagnosis, treatment and/or outcome history. Biological samples may be collected by any non-invasive means, such as, for example, by drawing blood from a subject, or using fine needle aspiration or needle biopsy. In certain embodiments, the biological sample is a serologic sample and is selected from the group consisting of whole blood, serum, plasma.

In certain embodiments, the one or more RA markers comprises C-Reactive Protein (CRP), Rheumatoid Factor (RF), Erythrocyte Sedimentation Rate (ESR), or anti-citrullinated peptide antibodies (ACPA). In some aspects, CRP is produced by the liver with levels of expression increasing in inflammatory conditions. RF is comprised of autoantibodies, such as IgM, that are specific for the Fc region of immunoglobulin G (IgG). ESR is a measure of how quickly red blood cells settle in a tube, with a higher ESR indicating faster settling. In some aspects, ESR is a non-specific indicator of inflammation. ACPAs are autoantibodies specific for proteins in which arginine amino acids have converted into citrulline residues. In embodiments where a serological covariate is obtained, one or more RA markers as discussed herein may be quantified. In further embodiments, at least two RA markers may be quantified. In even further embodiments, at least three RA markers may be quantified. In even further embodiments, at least four RA markers may be quantified. In some aspects, the serological covariates obtained comprise CRP, RF, ESR, and ACPA.

In certain aspects, increased levels of the serological markers discussed herein may be increased in many, but not all, patients with RA. In some aspects, individuals without RA have been observed to have increased levels of the serological markers discussed herein. In some respects, increased levels of serological markers may be associated with RA, however, have insufficient sensitivity and specificity alone to be diagnostic of RA.

In certain embodiments, in addition to the TUV values, changes in clinical presentation are assessed. In some embodiments, the one or more covariates obtained comprise a clinical covariate. A clinical covariate may be obtained from results of one or more clinical assessment tests. In certain implementations, the clinical covariate comprises the Health Assessment Questionnaire—Disease Index (HAQ-DI), the Clinical Disease Activity Index (CDAI), the Disease Activity Score of 28 Joints (DAS), and/or the Visual Analog Scale (VAS).

In some aspects, an individual, patient reported measure of disability in RA patients is the Health Assessment Questionnaire Disability Index (HAQ-DI). HAQ-DI scores represent physical function in terms of the patient's reported ability to perform everyday tasks, including the level of difficulty they experience in carrying out the activity. For example, the HAQ-DI may ask questions with regard to the patient's ability to perform tasks related to dressing, arising, eating, walking, hygiene, reach, grip, and other related activities. The questionnaire may also seek information about the patient's experience with pain. By recording the patients' ability to perform everyday activities, the HAQ-DI score can be used as one measure of their quality of life.

In some aspects, the CDAI is obtained by a physician in evaluating a patient's joints for evidence of swelling and tenderness for the 28 joints in the DAS evaluation, and further determining the number of joints that are either swollen or tender. The patient with RA and the physician separately and subjectively assess the patient's global disease activity. The CDAI score is a composite measure comprising the patient's swollen joint count (SJC), tender joint count (TJC), the patient's global assessment, and the physician's global assessment of the patient's disease activity.

In some aspects, the DAS is calculated by a medical practitioner based on various validated measures of disease activity, including physical symptoms of RA. A reduction in DAS reflects a reduction in disease severity. DAS28 is the Disease Activity Score in which 28 joints in the body are assessed to determine the number of tender joints and the number of swollen joints (Prevoo et al. Arthritis Rheum 38:44-48 1995). Twenty-two of these joints occur in the hands and wrists of the RA patient. The physician calculates the patient's SJC (swollen joint count) and TJC (tender joint count), and a serology test is performed. In the examples of the present disclosure, the serology test performed utilized the erythrocyte sedimentation rate (ESR). In other implementations of the DAS28 test, the serological marker used is the serum concentration of C-reactive protein (CRP). In some aspects, the physician and the patient may also jointly derive a subjective measure of the patient's global health (GH, scale of 1-100). In such implementations, an example of a DAS28-ESR may be calculated as: DAS28-ESR=0.56×sqrt(TJC)+0.28×sqrt (SJC)+0.70×1n(ESR)+0.014×GH.

In some aspects, the VAS is a validated and subjective measure for acute and chronic pain.

In embodiments where a clinical covariate is obtained, one or more clinical assessments may be conducted. In further embodiments, at least two clinical assessments may be conducted. In even further embodiments, at least three clinical assessments may be conducted. In even further embodiments, at least four clinical assessments may be conducted. In some aspects, the clinical covariates obtained comprise the HAQ-DI, CDAI, DAS, and VAS.

In some embodiments, the one or more covariates may comprise at least one serological covariate and at least one clinical covariate. In aspects, the one or more covariates are used in combination with at least one TUV_{Global} value for statistical modeling analysis. In other embodiments, the one or more covariates may comprise at least two covariates from the disclosed serological covariates and/or clinical covariates. In further embodiments, the one or more covariates may comprise at least three covariates from the disclosed serological covariates and/or clinical covariates. In even further embodiments, the one or more covariates may comprise at least four covariates from the disclosed serological covariates and/or clinical covariates.

Predicting Likelihood of Response Failure and/or Success

The disclosed methods are useful in predicting whether a subject with RA is likely to succeed or fail in a new course of RA therapy. That is, data from the subject's TUV values and one or more covariates are analyzed to determine whether the subject is likely to respond or not respond to a proposed new course of RA therapy. In aspects, the one or more covariates comprise one or more of a serological covariate and/or a clinical covariate. In some implementations, the disclosed methods are useful in determining whether a subject with RA will be likely to respond to a new RA therapy. In further embodiments, the disclosed methods are useful to determine whether a subject with RA is likely to fail to respond to a new RA therapy. In yet further embodiments, the disclosed methods are useful in allowing for early termination of a new RA therapy or treatment in a subject with RA by determining that further treatment is likely to fail. In further embodiments, the disclosed methods comprise the administration of an RA therapy to a subject based on the likelihood of treatment response to a new RA therapy. In some embodiments, the RA therapy comprises an aTNF therapy or non-aTNF therapy depending on the likelihood of treatment response to the aTNF therapy. In other embodiments, the RA therapy comprises an anti-TNF therapy, anti-IL6 therapy, anti-IL1 therapy, anti-CD20 therapy, anti-GM-CSF therapy, CTLA4-based therapies (such as, for example, abatacept), JAK inhibitors, or a combination thereof.

The American College of Rheumatology (ACR) proposed a set of criteria for classifying RA. The commonly used criteria are the ACR 1987 revised criteria (Arnett et al. Arthritis Rheum. 31:315-324 1988). Diagnosis of RA according to the ACR criteria requires a patient to satisfy a minimum number of listed criteria, such as tender or swollen joint counts, stiffness, pain, radiographic indications and measurement of serum rheumatoid factor. In some aspects, the likelihood of treatment response is the likelihood that the RA therapy results in an at least 20% reduction in an ACR criteria score of the subject at about 24 weeks after the administration of the RA therapy. In further aspects, the likelihood of treatment response is the likelihood that the RA therapy results in an at least 50% reduction in an ACR criteria score of the subject at about 24 weeks after the administration of the RA therapy. In alternative aspects, the likelihood of treatment response is the likelihood that the RA therapy results in an at least 20%, at least 30%, at least 40%, at least 50%, at least 60%, at least 70%, at least 80%, or at least 90% reduction in the ACR criteria score of the subject at about 24 weeks after the administration of the RA therapy.

In some aspects wherein the treatment administered is the RA therapy, the RA therapy results in an at least 20% reduction, at least 30% reduction, at least 40% reduction, at least 50% reduction, at least 60% reduction, at least 70% reduction, at least 80% reduction, or at least 90% reduction in the ACR criteria score of the subject at about 24 weeks after the administration of the RA therapy. In implementations, the treatment administered is an aTNF therapy, and the aTNF therapy results in an at least 20% reduction, at least 30% reduction, at least 40% reduction, at least 50% reduction, at least 60% reduction, at least 70% reduction, at least 80% reduction, or at least 90% reduction in the ACR criteria score of the subject at about 24 weeks after the administration of the aTNF therapy.

In certain implementations, a subject's TUV_{Global} values are used in conjunction with one or more covariates comprising a serological covariate and/or a clinical covariate as part of a multivariate statistical model to determine the probability or likelihood the subject will respond to a new RA therapy (e.g. an aTNF therapy or DMARD therapy). In exemplary implementations, the statistical modeling comprises a logistic regression model, such as a multivariate logistical regression, to predict the likelihood of treatment response to an RA therapy. In further embodiments, alternative statistical modeling may be used, including, but not limited to, artificial neural networks or other machine learning techniques, decision trees, and support vector machines. In further aspects, quantitative methods for image analysis other than determining the TUV values may also be applicable. In further aspects, imaging agents other than Tc99m tilmanocept may be used as discussed herein to combine imaging outputs with clinical assessments and/or serology markers to build models that accurately predict RA treatment responses.

After applying a statistical modeling to the at least one TUV_{Global} value in conjunction with one or more covariates comprising a serological covariate and/or a clinical covariate, the likelihood of response to the RA therapy is determined. As discussed herein, the RA therapy may comprise an anti-TNF (aTNF) therapy, anti-IL6 therapy, anti-IL1 therapy, anti-CD20 therapy, anti-GM-CSF therapy, CTLA4-based therapy, JAK inhibitors, other DMARD therapy, or a combination thereof. In some aspects, a treatment is administered to the patient/subject based on the likelihood of response for the evaluated RA therapy using statistical modeling. In embodiments, the treatment is a treatment for RA. In some examples, the treatment may comprise the RA therapy evaluated under statistical modeling, or may comprise a different RA therapy that is not the RA therapy evaluated under statistical modeling. In embodiments, the treatment comprises an RA therapy comprising an anti-TNF (aTNF) therapy, anti-IL6 therapy, anti-IL1 therapy, anti-CD20 therapy, anti-GM-CSF therapy, CTLA4-based therapy, or JAK inhibitors, and wherein the treatment may be the same RA therapy or may be a different RA therapy described herein that is not the RA therapy assessed under the statistical modeling.

In some examples, the RA therapy assessed by statistical modeling is the aTNF therapy. Based on the likelihood of response to the aTNF therapy, the treatment administered to the subject may be the aTNF therapy, or may comprise an RA therapy that is not the aTNF therapy, such as, anti-IL6 therapy, anti-IL1 therapy, anti-CD20 therapy, anti-GM-CSF therapy, CTLA4-based therapy, JAK inhibitors, or a combination thereof. In similar examples, the RA therapy assessed under the statistical modeling to determine a likelihood of response may comprise any one of an anti-TNF (aTNF) therapy, anti-IL6 therapy, anti-IL1 therapy, anti-CD20 therapy, anti-GM-C SF therapy, CTLA4-based therapy, or JAK inhibitors. In such examples, based on the likelihood of response to the RA therapy assessed under statistical modeling, the treatment administered to the subject may be the RA therapy assessed under statistical modeling, or one of the other RA therapies described herein that is not the RA therapy assessed under statistical modeling.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

The following study was conducted with 28 evaluable subjects with RA that were initiating a new aTNF therapy. Subjects underwent planar gamma camera imaging following intravenous (IV) administration of 150 μg of TC99m tilmanocept (Lymphoseek®) labeled with 10mCi of 99mtechnetium prior to initiating their new aTNF therapy and again at 5, 12, and 24 weeks after treatment initiation (i.e. each subject was imaged 4 times—had 4 imaging events). At each imaging event, subjects underwent a clinical evaluation of their RA disease activity that included determinations of their 28 joint disease activity score (DAS28) and their clinical disease activity index (CDAI). Subjects were also evaluated for the American College of Rheumatology (ACR) disease activity score. Subjects were deemed to have responded to their newly initiated aTNF therapy if their ACR score had declined by 50% or more relative to their score before initiation of therapy (ACR50 response). Also, at each imaging event, subjects had blood drawn for evaluation of various blood markers that included ACPA, rheumatoid factor (RF), and c-reactive protein levels (CRP). ACPA levels at baseline (prior to initiation of the new aTNF therapy) fell into two distinct groups: one with ACPA levels≤30, and a second group with ACPA levels>190, with few subjects with ACPA levels between these 2 groups. ACPA levels before initiation of the new aTNF therapy: a) ACPA levels≤80 predicts treatment failure (non-response); and b) ACPA Level>80 predicts treatment success (significant clinical improvement).

For DAS28 and CDAI, positive clinical responses were determined by significant declines in the respective scores at weeks 12 or 24 relative to the scores at baseline (prior to initiation of the new therapy). The designation of significant declines in DAS28 or CDAI were determined in reference to the medical literature.

### Tc99M Tilmanocept Localization to Ra Inflamed Joints—Quantification by the Global TUV Method

There are two ways to parse the Global TUV data:

TUV_{Global-Δ5wk}: If Global TUV declines≥10% between baseline (Day0) and week 5, then the prediction is that the patient will respond (clinically improve) at week 12 or 24 compared to baseline. TUV_{Global-Δ5wk} declines of <10% or increases in Global TUV at week 5 compared to baseline predict treatment failure.
Bucket method: Subjects with baseline Global TUVs≤4.00 are predicted to fail. For subjects with baseline Global TUVs>4.00, the TUV_{Global-Δ5wk} declines of ≥10% between baseline (Day0) and week 5 predict treatment success.

All examples are based on analyses of 28 subjects with RA that were initiating new aTNF therapies. 14 (50%) and 15 (53.6%) subjects experienced a response (clinical improvement) at week 24 by DAS28 and CDAI respectively. Five (18%) subjects experienced an ACR50 or better response.

Bucket Method plus ACPA

Subjects evaluated by the Bucket Method for TUV_{Global}-DO (Baseline Global TUV) and TUV_{Global-Δ5wk}. The ACPA method was also used. Results are displayed in Table 1(A-I) for the performances of TUV_{Global-Δ5wk} alone, ACPA alone, and the Bucket Method plus ACPA. In the bucket method plus ACPA, if the ACPA was >80 then the prediction is treatment success and in subjects with TUV_{Global}-Do>4.00, a TUV_{Global-Δ5wk} decline of ≥10% predicts success. The results are displayed for week 24 clinical results as truth tables and descriptive statistics.

Truth Tables

TUV (Buckets) Compared to Clinical Outcomes

TABLE 1A
ACR50

| | Clin+ | Clin− | |
|---|---|---|---|
| TUV+ | 2 | 2 | 4 |
| TUV− | 3 | 21 | 24 |
| | 5 | 23 | |

| | |
|---|---|
| Sens | 0.400 |
| Spec | 0.913 |
| PPV | 0.500 |
| NPV | 0.875 |
| Accuracy | 0.821 |

TABLE 1B
DAS28

| | Clin+ | Clin− | |
|---|---|---|---|
| TUV+ | 4 | 0 | 4 |
| TUV− | 10 | 14 | 24 |
| | 14 | 14 | |

| | |
|---|---|
| Sens | 0.286 |
| Spec | 1.000 |
| PPV | 1.000 |
| NPV | 0.583 |
| Accuracy | 0.643 |

TABLE 1C
CDAI

| | Clin+ | Clin− | |
|---|---|---|---|
| TUV+ | 4 | 0 | 4 |
| TUV− | 11 | 13 | 24 |
| | 15 | 13 | |

| | |
|---|---|
| Sens | 0.267 |
| Spec | 1.000 |
| PPV | 1.000 |
| NPV | 0.542 |
| Accuracy | 0.607 |

Tables 1A-1C: Truth Tables for TUV_{Global-Δ5wk} Only for Predicting Treatment Outcomes. NPV is the probability that a subject predicted to fail aTNF therapy actually failed therapy at 24 weeks after initiating aTNF therapy. Sensitivity (Sens) is the proportion of subjects who actually responded by week 24 who were predicted to have responded by the test.

Truth Tables

ACPA Compared to Clinical Outcomes

TABLE 1D
ACR50

| | Clin+ | Clin− | |
|---|---|---|---|
| ACPA+ | 4 | 8 | 12 |
| ACPA− | 1 | 15 | 16 |
| | 5 | 23 | |

| | |
|---|---|
| Sens | 0.800 |
| Spec | 0.652 |
| PPV | 0.333 |
| NPV | 0.938 |
| Accuracy | 0.679 |

TABLE 1E
DAS28

| | Clin+ | Clin− | |
|---|---|---|---|
| ACPA+ | 9 | 3 | 12 |
| ACPA− | 5 | 11 | 16 |
| | 14 | 14 | |

| | |
|---|---|
| Sens | 0.643 |
| Spec | 0.786 |
| PPV | 0.750 |
| NPV | 0.688 |
| Accuracy | 0.714 |

TABLE 1F
CDAI

| | Clin+ | Clin− | |
|---|---|---|---|
| ACPA+ | 9 | 3 | 12 |
| ACPA− | 6 | 10 | 16 |
| | 15 | 13 | |

| | |
|---|---|
| Sens | 0.600 |
| Spec | 0.769 |
| PPV | 0.750 |
| NPV | 0.625 |
| Accuracy | 0.679 |

Tables 1D-1F: Truth Tables for ACPA Only for Predicting Treatment Outcomes. NPV is the probability that a subject predicted to fail aTNF therapy actually failed therapy at 24 weeks after initiating aTNF therapy. Sensitivity (Sens) is the proportion of subjects who actually responded by week 24 who were predicted to have responded by the test.

Truth Tables

TUV (Buckets) and ACPA (T&AC1 Compared to Clinical Outcomes

TABLE 1G
ACR50

| | Clin+ | Clin− | |
|---|---|---|---|
| T&AC+ | 5 | 9 | 14 |
| T&AC− | 0 | 14 | 14 |
| | 5 | 23 | |

| | |
|---|---|
| Sens | 1.000 |
| Spec | 0.609 |
| PPV | 0.357 |
| NPV | 1.000 |
| Accuracy | 0.679 |

TABLE 1H
DAS28

| | Clin+ | Clin− | |
|---|---|---|---|
| T&AC+ | 11 | 3 | 14 |
| T&AC− | 3 | 11 | 14 |
| | 14 | 14 | |
| Sens | 0.786 | | |
| Spec | 0.786 | | |
| PPV | 0.786 | | |
| NPV | 0.786 | | |
| Accuracy | 0.786 | | |

TABLE 1I
CDAI

| | Clin+ | Clin− | |
|---|---|---|---|
| T&AC+ | 11 | 3 | 14 |
| T&AC− | 4 | 10 | 14 |
| | 15 | 13 | |

| | |
|---|---|
| Sens | 0.733 |
| Spec | 0.769 |
| PPV | 0.786 |
| NPV | 0.714 |
| Accuracy | 0.750 |

Tables 1G-1I: Truth Tables for ACPA Only for Predicting Treatment Outcomes. NPV is the probability that a subject predicted to fail aTNF therapy actually failed therapy at 24 weeks after initiating aTNF therapy. Sensitivity (Sens) is the proportion of subjects who actually responded by week 24 who were predicted to have responded by the test.

The utility of the disclosed invention is that it enables physicians to identify more quickly (within 5 weeks) those RA patients initiating a new aTNF therapy who will not respond adequately to their new therapy, permitting these patients to be moved to an alternative therapy that has a greater chance of providing an effective response. The key feature of this test is that it must have a high negative predictive value (NPV) and sensitivity to predict response. Stated differently, the test should minimize the number of patients predicted to fail therapy who actually would go on to respond to their therapy should it be continued. Such patients if switched to an alternative therapy, which itself could fail, would be denied the benefits they would have accrued from continuing their recently initiated aTNF therapy. Physicians commonly use the ACR, DAS28, and CDAI to evaluate their RA patients to determine if they are responding to a new therapy, however, it takes up to 6 months for the benefits of a new therapy to fully manifest. The disclosed test that combines information from both TUV_{global} and ACPA provides a tool to facilitate an earlier determination of treatment success. In Tables 1A through 1I, regardless of whether a physician uses ACR, DAS28 or CDAI as their preferred clinical test, it is shown that the combined (TUV_{Global}+ACPA) had higher sensitivities and NPV for treatment response than either TUV_{global} or ACPA used alone. The example of the ACR50 response is remarkable in that the combined TUV_{global} and ACPA test had both a sensitivity and a NPV of 100%, meaning that no RA patient that would have been advised to switch their treatment as a result of this test would have experienced a treatment response had they remained on the aTNF therapy. Concurrently, half (14 of 28) subjects in this disclosed study could have been switched early to an alternative therapy without risking losing an opportunity to benefit from aTNF therapy.

### Example 2

A clinical study was further conducted to evaluate the ability to determine more quickly the probability of whether a patient with RA was going to respond to a new RA therapy by utilizing statistical modeling and additional serological and clinical covariates. The clinical study evaluated 27 subjects with RA who were initiating a new treatment with a tumor necrosis factor specific antibody (i.e., a new anti-TNF therapy).

The study involved collecting and analyzing planar gamma images of the hands and wrists of the subjects using Tc99m tilmanocept as the imaging agent. The hands and wrists of the subjects in this study were imaged twice by planar gamma imaging: once prior to initiating their new anti-TNF therapy (TO), and again 5 weeks after initiation of therapy (T5wks). The image analyses consisted of calculating the global MARTAD values (or TUV_{Global}) for each image. Global MARTAD values derived from planar gamma images of the hands and wrists of RA patients were used to assess the macrophage involvement in the pathobiology of the individual subjects. At T0, various clinical assessments performed routinely on RA subjects by the rheumatologists managing their care were performed.

At T0, a series of clinical laboratory serology tests were performed on all subjects. Subjects also underwent clinical assessments 12 and 24 months after initiation of their new anti-TNF therapy. The goal of the study was to obtain preliminary results showing that global MARTAD results at T0 and the difference in global MARTAD results from T0 to T5wks in combination with clinical assessments and serological results at T0 could predict clinical outcomes of the new anti-TNF therapy at weeks 12 and/or 24, thus accelerating the determination of the effectiveness of a new anti-TNF therapy.

The example combined MARTAD values (T0 and change at week 5) with clinical assessments and serological findings from T0 in logistic regression models to determine if such models could accurately identify those subjects that would achieve an ACR50 response at week 24.

Logistical regression models were constructed that evaluated different combinations of the MARTAD values (T0 and T5wks), clinical assessments, and serological markers as independent covariates. The outputs of the models were their abilities to predict treatment response to a new anti-TNF therapy as measured by an ACR50 or better response at 24 weeks. The results are shown below in Tables 2, 3 and 4. Table 2 provides the results of using TUV_{Global} values combined with serological covariates, Table 3 provides the results of using TUV_{Global} values combined with clinical assessment covariates, and Table 4 provides the results of using TUV_{Global} values combined with a mixture of serological and clinical assessment covariates. The outputs for the various models are displayed as areas under receiver operating characteristic curves (ROC curves). Areas under ROC curves (AUCs) of 1.0 indicate perfect prediction of responses and nonresponses. AUC values of 0.5 indicate that the model had no predictive capabilities.

TABLE 2
TUV_{Global} Values Combined with Serological Covariates

| Model | | | |
|---|---|---|---|
| Number | Co-variates | Response | AUC |
| 1 | T0-TUV, T5-TUV, CRP, | Wk24-ACR50 | 0.9000 |
| | RF, ESR, ACPA | | |
| 2 | T0-TUV, T5-TUV | Wk24-ACR50 | 0.5364 |
| 3 | CRP, RF, ESRN, ACPA | Wk24-ACR50 | 0.7714 |
| 4 | T0-TUV, T5-TUV, CRP | Wk24-ACR50 | 0.7636 |
| 5 | T0-TUV, T5-TUV, RF | Wk24-ACR50 | 0.7909 |
| 6 | T0-TUV, T5-TUV, ACPA | Wk24-ACR50 | 0.6455 |
| 7 | T0-TUV, T5-TUV, ESR | Wk24-ACR50 | 0.7619 |

As shown in Table 2, combining TUV_{Global} values obtained before initiation of a new anti-TNF therapy (T0-TUV), the difference between TUV values obtained before initiation of a new anti-TNF therapy and after 5 weeks of therapy (T5-TUV), and serological values observed before initiation of the new anti-TNF therapy (CRP, RF, ESR, ACPA) enabled creation of a logistic regression model (Table 2, Model 1) with an ROC curve AUC of 0.9000, indicating that the model had a high discriminatory accuracy for identifying those study subjects that would and would not achieve an ACR50 or better response observed after 24 weeks of treatment. The remaining 6 models described in Table 2 examined the components of Model 1 separately. Models 2 and 3 evaluated the TUV_{Global} values (T0-TUV and T5-TUV, AUC 0.5364) and the combined four serological markers (AUC, 0.7714) respectively. While the AUC for the TUV_{Global} values alone were modest, these results show that combining the TUV_{Global} values with the serological makers markedly increased the discriminatory accuracy of the model with the combined serological markers without TUV_{Global} values included. The remaining models showed that all the serological marker individually increased the AUC of models that included the TUV_{Global} values.

TABLE 3
TUV_{Global} Values Combined with Clinical Covariates

| Model | | | |
|---|---|---|---|
| Number | Co-variates | Response | AUC |
| 1 | T0-TUV, T5-TUV, | Wk24-ACR50 | 1.0000 |
| | HAQ-DI, CDAI, DAS, | | |
| | VAS | | |
| 2 | T0-TUV, T5-TUV | Wk24-ACR50 | 0.5364 |
| 3 | HAQ-DI, CD AI, DAS, VAS | Wk24-ACR50 | 0.8273 |
| 4 | T0-TUV, T5-TUV, HAQ-DI | Wk24-ACR50 | 0.8273 |
| 5 | T0-TUV, T5-TUV, CDAI, | Wk24-ACR50 | 0.5545 |
| 6 | T0-TUV, T5-TUV, DAS | Wk24-ACR50 | 0.5727 |
| 7 | T0-TUV, T5-TUV, VAS | Wk24-ACR50 | 0.7455 |

Table 3 shows the ROC curves AUCs of models that combined TUV_{Global} values with clinical assessment results obtained prior to initiation of the new anti-TNF therapies. Model 1 in Table 3 shows the predictive ability of a model that combined TUV_{Global} values with all four pretreatment clinical assessments (HAQ-DI, CDAI, DAS, VAS). The ROC curve AUC for Model 1 on Table 3 was a remarkable 1.000, indicating that this model could predict ACR50 or better responses observed after 24 weeks of treatment with 100% accuracy in the evaluated data set). As with the results shown in Table 2, the remaining results shown in Table 3 evaluated the components of Model 1 (Table 3) separately or combined individually with TUV_{Global} values. Model 3 in Table 3 shows the results of a model constructed with just the four clinical assessment covariates. This model produced an ROC curve with an AUC of 0.8857, which while positive, was further improved by adding the TUV_{Global} values. Interestingly, the ROC curve constructed with the TUV_{Global} values combined with just the pretreatment HAQ-DI values also produced an AUC of 0.8857, suggesting that the TUV_{Global} values contributed as much independent information as did the other three clinical assessments when combined with HAQ-DI values.

TABLE 4
TUV_{Global} Values Combined with Mixed Covariates

| Model | | | |
|---|---|---|---|
| Number | Co-variates | Response | AUC |
| 1 | T0-TUV, T5-TUV, | Wk24-ACR50 | 0.9714 |
| | ESR, RF, HAQ-DI | | |
| 2 | T0-TUV, ESR, RF, | Wk24-ACR50 | 0.8857 |
| | HAQ-DI (no T5-TUV) | | |
| 3 | T0-TUV, T5-TUV | Wk24-ACR50 | 0.5364 |
| 4 | ESR, RF | Wk24-ACR50 | 0.7048 |
| 5 | T0-TUV, T5-TUV, HAQ-DI | Wk24-ACR50 | 0.8273 |
| 6 | T0-TUV, T5-TUV, ESR | Wk24-ACR50 | 0.7619 |
| 7 | T0-TUV, T5-TUV, RF | Wk24-ACR50 | 0.7909 |

Table 4 shows the results of a model constructed with TUV_{Global} values combined with ESR, RF, and HAQ-DI values obtained before initiation of the new anti-TNF therapy (Model 1, Table 4). The ROC curve AUC for this model was 0.9714, which is close to the AUC of 1.000 for Model 1 on Table 3 and greater than the 0.9000 AUC observed for the four serologic markers combined with the MARTAD values (Model 1, Table 2). This model combining TUV_{Global} values, ESR, RF and HAQ-DI was constructed to show that a model with high discriminatory accuracy to predict ACR50 or better responses can be constructed with a mixture of serological and clinical co-variates and TUV_{Global} values. Further shown in Table 4 are the results of a model constructed with the same covariates as Model 1 except that the values for the difference in TUV_{Global} values between T0 and week 5 (T5-TUV) were left out (Model 2). From Model 2, it is shown that the AUC of Model 1 is reduced to 0.8857 by leaving out the T5-TUV values, indicating that the difference in TUV_{Global} values between T0 and week 5 contributed to the discriminatory ability of Model 1.

Of the 27 subjects evaluated, 5 subjects had an ACR50 or better response after 24 weeks of treatment. The results indicate that the disclosed methods utilizing the combination of TUV_{Global} values and various covariates in a statistical model are sufficient to construct more robust treatment prediction models for patients with RA undergoing RA therapy. Further, similar techniques may be used to predict RA treatment responses at other times after initiation of a new therapy which are not limited to anti-TNF therapy. Examples of such other clinically useful time points for which prediction of clinical responses may be made, include 3 months after initiation of the new therapy and 1 year after initiation of a new therapy.

The disclosures being thus described, it will be obvious that the same may be varied in many ways. Such variations are not to be regarded as a departure from the spirit and scope of the disclosures and all such modifications are intended to be included within the scope of the following claims.

What is claimed is:

1. A method of treating a subject with rheumatoid arthritis (RA) comprising:
a. administering to the subject a composition comprising a macrophage targeting construct and an imaging moiety conjugated thereto;
b. acquiring planar images of a plurality of joints of the subject;
c. determining at least one TUV_{Global} value for the subject from the planar images;
d. obtaining one or more covariates comprising:
i) a serological covariate obtained from a serum sample from the subject and quantifying the level of one or more RA markers in the serum; and/or
(ii) a clinical covariate obtained from results of one or more clinical assessment tests;
e. applying statistical modeling to the at least one TUV_{Global} and the one or more covariates to determine a likelihood of response to an RA therapy; and
f. administering a treatment to the subject based on the likelihood of response to the RA therapy.

2. The method of claim 1, wherein the RA therapy comprises an anti-TNF (aTNF) therapy, anti-IL6 therapy, anti-IL1 therapy, anti-CD20 therapy, anti-GM-CSF therapy, CTLA4-based therapy, JAK inhibitors, or a combination thereof.

3. The method of claim 2, wherein the treatment comprises the RA therapy evaluated from step (e), or an RA therapy excluding the RA therapy evaluated from step (e).

4. The method of claim 1, wherein the RA therapy comprises an aTNF therapy, and wherein the treatment comprises the aTNF therapy or a non-aTNF therapy.

5. The method of claim 1, wherein the serological covariate comprises C-Reactive Protein (CRP), Rheumatoid Factor (RF), Erythrocyte Sedimentation Rate (ESR), or anti-citrullinated peptide antibodies (ACPA).

6. The method of claim 1, wherein the clinical covariate comprises the Health Assessment Questionnaire—Disease Index (HAQ-DI), Clinical Disease Activity Index (CDAI), Disease Activity Score of 28 Joints (DAS), or Visual Analog Scale (VAS).

7. The method of claim 5, wherein the CRP, RF, ESR, and ACPA are obtained.

8. The method of claim 6, wherein the HAQ-DI, CDAI, DAS, and VAS are obtained.

9. The method of claim 1, wherein at least one serological covariate and at least one clinical covariate are obtained.

10. The method of claim 1, wherein the at least one TUV_{Global} value is determined prior to the administration of the RA therapy.

11. The method of claim 1, wherein the at least one TUV_{Global} value is determined at a time period between one week and 24 weeks after the administration of the RA therapy.

12. The method of claim 1, wherein the likelihood of treatment response is the likelihood that the RA therapy results in an at least 20% reduction in an ACR criteria score (American College of Rheumatology/European League Against Rheumatism 2010 criteria) of the subject at about 24 weeks after the administration of the RA therapy.

13. The method of claim 2, wherein the RA therapy administered is the aTNF therapy and results in an at least 50% reduction in an ACR criteria score (American College of Rheumatology/European League Against Rheumatism 2010 criteria) of the subject at about 24 weeks after the administration of the RA therapy.

14. The method of claim 2, wherein the RA therapy administered is the aTNF therapy and results in an at least 70% reduction in an ACR criteria score (American College of Rheumatology/European League Against Rheumatism 2010 criteria) of the subject at about 24 weeks after the administration of the RA therapy.

15. The method of claim 1, wherein the statistical modeling comprises a logistic regression model.

16. The method of claim 1, wherein the step of determining the TUV_{Global} value further comprises:
a. selecting a plurality of joints in the subject where inflammation is suspected;
b. acquiring one or more planar images of each of the plurality of joints;
c. for each joint image, defining a region of interest (ROI) comprising the joint;
d. for each joint, defining a joint specific reference region (RR);
e. for each joint, determining a TUV_{Joint} value of the joint by assessing the ratio of average pixel intensity of the ROI to the average pixel intensity of the RR;
f. for each joint, comparing the TUV_{Joint} value of the joint to a normal TUV_{Joint} value for a corresponding joint, wherein the normal TUV_{Joint} value is derived from averaging the TUV_{Joint} values for the corresponding joint from a plurality of healthy subjects, and wherein macrophage involvement is indicated by a joint specific TUV_{Joint} value that exceeds the normal TUV_{Joint} value by a predetermined threshold;
g. for each joint having a joint specific TUV_{Joint} value that exceeds the normal TUV_{Joint} value by a predetermined threshold, calculating a macrophage-involved contribution (MI) of the joint by dividing the difference of the TUV_{Joint} and normal TUV_{Joint} by the normal TUV_{Joint}; and
h. determining the TUV_{Global} value for the subject by determining the sum of the MI for all of the joints of the subject that exceeds the predetermined threshold.

17. The method of claim 1, wherein the macrophage targeting construct is a mannosylated dextran construct comprising Tc99m-tilmanocept, and wherein the quantity of Tc99m-tilmanocept administered is between about 50 μg and about 400m.

18. The method of claim 1, wherein the subject is initiating a new RA therapy.

19. The method of claim 18, wherein the method is performed prior to the subject initiating a new RA therapy.

20. A method of predicting a subject's likelihood of response to a new RA therapy comprising:
a. administering to the subject a composition comprising a macrophage targeting construct and an imaging moiety conjugated thereto;
b. acquiring planar images of a plurality of joints of the subject;
c. determining at least one TUV_{Global} value for the subject from the planar images;
d. obtaining one or more covariates comprising:
i) a serological covariate obtained from a serum sample from the subject and quantifying the level of one or more RA markers in the serum; and/or
(ii) a clinical covariate obtained from results of one or more clinical assessment tests;
e. applying statistical modeling to the at least one TUV_{Global} and the one or more covariates to determine the likelihood of treatment response to a new anti-TNF (aTNF) therapy.
